(19) United States
(12) Patent Application Publication
(10) Pub. No.: US 2002/0090404 A1
(43) Pub. Date: Jul. 11, 2002

(54) Components of canola for treating hyperlipidemia

(76) Inventors: Najla Guthrie, London (CA); Elzbieta Maria Kurowska, London (CA)

Correspondence Address: Davidson, Davidson & Kappel, LLC, 14th Floor, 485 Seventh Avenue, New York, NY, 10018, US

(21) Appl. No.: 09/952,477

(22) Filed: Sep. 14, 2001

Related U.S. Application Data
(60) Provisional application No. 60/232,934, filed on Sep. 15, 2000.

(51) Int. Cl. A61K 35/78; A61K 31/70

(52) U.S. Cl. 424/755; 514/23

(57) ABSTRACT

Disclosed is a pharmaceutical composition comprising at least one canola extract effective in reducing hyperlipidemia and a pharmaceutically acceptable excipient. Also disclosed are methods of use thereof.

This application claims priority from U.S. Provisional Application No. 60/232,934, filed Sep. 15, 2000, hereby incorporated by reference.

### FIELD OF THE INVENTION

The present invention relates to methods and compositions for treating atherosclerosis and hypercholesterolemia with canola extracts.

### BACKGROUND OF THE INVENTION

Hyperlipidemia is a pathological state in mammals, where there is an abnormally high concentration of lipids circulating in the serum. The composition of the lipid pool in the circulation consists mostly of triglyceride (fatty acid esters of glycerol), cholesterol, and fatty acid esters of cholesterol. Such molecules are generally found bound to specific proteins in the form of complexes which act as transporting mechanisms. Hyperlipidemia is a condition which is commonly associated with elevated levels of cholesterol, phospholipids, and/or triglycerides in the blood serum of mammals.

The hyperlipidemias include six types of inheritable hyperlipoproteinemias; these types frequently are referred to as lipoprotein phenotypes. The major plasma lipids, including cholesterol and the triglycerides do not circulate freely in solution in plasma, but are bound to proteins and transported as macromolecular complexes called lipoproteins. Classification of inherited hyperlipoproteinemias according to phenotype is important, since dietary management and drug therapy are largely dependent on this information. (The Merck Manual, 16^{th} edition, Robert Berkow and Andrew J. Fletcher, Merck & Co., Inc., Rahway, N.J. 1992). In the current practice of treating hyperlipidemia the goal is to lower lipid levels by weight control and diet control. As an adjunct to diet and weight control, blood lipid reducing agents, including, e.g., prescription drugs, may also be administered.

Plasma lipoproteins are carriers of lipids from the sites of synthesis and absorption to the sites of storage and/or utilization. Lipoproteins are spherical particles with triglycerides and cholesterol esters in their core and a layer of phospholipids, nonesterified cholesterol and apolipoproteins on the surface. Lipoproteins are categorized into five major classes based on their hydrated density as very large, triglyceride-rich particles known as chylomicrons, very low density lipoproteins (VLDL), intermediate-density lipoproteins (IDL), low-density lipoproteins (LDL) and, high-density lipoproteins (HDL).

Apolipoproteins are protein components of lipoproteins with three major functions which include: (1) maintaining the stability of lipoprotein particles, (2) acting as cofactors for enzymes that act on lipoproteins, and (3) removing lipoproteins from circulation by receptor-mediated mechanisms. The four groups of apolipoproteins are apolipoproteins A (Apo A), B (Apo B), C (Apo C) and E (Apo E).

LDL consists of a hydrophobic lipid core composed of cholesterol esters and triglycerides. The lipid core of the LDL particle is surrounded by an amphipathic coat composed of phospholipids, unesterified cholesterol and Apo B.

Several studies have shown that an increased Apo B level in blood is a reliable marker for coronary atherosclerosis (Sniderman, A. et al., Proc. Natl. Acad. Sci. USA, 77:604-608 (1980); Kwiterovich, P. O. et al., Am. J. Cardiol., 71:631-639 (1993); McGill et al. Coron. Artery Dis., 4:261-270 (1993); Tornvall, P. et al., Circulation, 88:2180-2189 (1993)).

In the United States, the complications of arteriosclerosis account for about one half of all deaths and for about one third of deaths in persons between 35 and 65 years of age. Atherosclerosis, or the development of atheromatous plaques in large and medium-sized arteries, is the most common form of arteriosclerosis. Many factors are associated with the acceleration of atherosclerosis, regardless of the underlying primary pathogenic change, for example, age, elevated plasma cholesterol level, high arterial blood pressure, cigarette smoking, reduced high-density lipoprotein (HDL) cholesterol levels, or family history of premature coronary artery disease.

The risk of death from coronary artery disease has a continuous and graded relation to total serum cholesterol levels greater than 180 mg/dl (Stamler, J. et al., (1986) JAMA 256:2823). Approximately one third of adults in the United States have levels that exceed 240 mg/dl and, therefore, have a risk of coronary artery disease that is twice that of people with cholesterol levels lower than 180 mg/dl. Acceleration of atherosclerosis is principally correlated with elevation of LDL, or beta fraction, has a negative correlation with atherosclerosis (Castelli, W. P. et al. (1986) JAMA 256:2835). HDL exerts a protective effect and the ratio of total cholesterol to HDL cholesterol is a better predictor of coronary artery disease than the level of either alone. Total cholesterol levels are classified as being desirable (<200 mg/dl), borderline high (200-239 mg/dl), or high (>240 mg/dl)(Report of the National Education Program Expert Panel on Detection, Evaluation, and Treatment of High Blood Cholesterol in Adults (1988) Arch Intern Med 148:36).

Advances in the study of cholesterol metabolism and coronary disease have initiated an era of increased emphasis on preventive therapy. New guidelines for the detection and treatment of high blood cholesterol in adults recommend that patients with high cholesterol levels or with borderline-high levels and two or more additional risk factors should have a measurement of LDL. LDL cholesterol levels are then classified as borderline high risk (130-159 mg/dl) or high risk (>160 mg/dl). Dietary treatment is recommended for those patients with high-risk levels of LDL and for those with borderline-high risk levels who have two or more additional risk factors. Drug treatment is recommended for all patients with LDL levels greater than 189 mg/dl and for those patients with LDL cholesterol levels between 159 and 189 mg/dl who have two or more additional risk factors.

In view of the above, it is not surprising to find that a number of compounds have been proposed for the treatment of hyperlipidemia in mammals. For example, colestipol hydrochloride (U.S. Pat. Nos. 3,692,895 and 3,803,237) is a basic anion exchange resin which, when ingested, sequesters bile acids in the intestine. This stimulates the production of bile acids, which uses and depletes the body's stored cholesterol. This in turn reduces LDL levels. Gemfibrozil, described in U.S. Pat. No. 3,674,836 is also used in such treatment. Niacin (3-pyridinecarboxylic acid) is also administered for hypercholesterolemia, at a dosage of about 1.5 to 6 g/day orally. Other pharmaceutical agents occasionally administered for hyperlipidemia include neomycin, norethindrone acetate, oxandrolone, and dextrothyroxine (Remington's Pharmaceutical Sciences, (17th Ed., Mack Pub. Co., 1985), pp. 863-865). U.S. Pat. No. 4,499,303 describes the use of a class of N-benzoylsulfamates and benzoylsulfonamides as useful hypolipidemic agents. U.S. Pat. No. 4,395,417 proposes the use of cyclic imides, diones, reduced diones and analogs as useful agents. All aforementioned references of which are herein incorporated by reference.

In spite of the numerous compounds and methods which have been proposed for the control of hyperlipidemia, the need remains for an agent having enhanced lowering of elevated serum lipoprotein lipids. By virtue of the present invention, it has been discovered that canola extracts can be utilized to lower lipoprotein levels.

Canola is a cruciferous crop which is mainly utilized for its extracted oil. After the oil has been extracted a protein rich meal remains which is used as a ruminant in animal diets. Further extraction of the canola meal yields minor components from canola, including, glucosinolates, phenolic acid esters and phenolic acids. The total content of selected minor components in Canola extracts are listed below:

| | mg/g extract | % content |
|---|---|---|
| Total glucosinates | 8.61 | 0.9% |
| (flaked, cooked Canola seeds) | | |
| Total phenolic acids | 134.00 | 13.4% |
| (flaked, cooked Canola seeds) | | |
| Total phenolic acids | 53.15 | 5.3% |
| (Canola meal) | | |
| Free phenolic acids | 246.64 | 24.7% |
| (Canola meal extract after | | |
| hydrolysis) | | |

* The remaining components of extracts are mostly sugars and small amounts and saponins.

Glucosinolates present in the extract from flaked, cooked canola seeds are listed below:

| | μM/g extract | mg/g extract |
|---|---|---|
| Progoitrin | 8.52 | 3.45 |
| Gluconapin | 5.89 | 2.29 |
| 4-hydroxybrassicin | 3.22 | 1.55 |
| Glucobrassicanapin | 0.90 | 0.36 |
| Glucoalyssin | 0.64 | 0.27 |
| Napoleiferin | 0.54 | 0.23 |
| Glucobrassicin | 0.40 | 0.19 |
| Glucoraphanin | 0.22 | 0.09 |
| Sinigrine | 0.19 | 0.07 |
| Gluconasturtin | 0.19 | 0.08 |
| Neoglucobrassicin | 0.06 | 0.03 |
| 4-methoxyglucobrassicin | traces | — |

Content of phenolic acids in the extract from canola meal (mg/g extract) are listed below:

| | Protocatechuic | Caffeic | p-coumaric | Ferulic | Sinapic |
|---|---|---|---|---|---|
| Free phenolic | Trace | 0.03 | 0.02 | 0.02 | 1.03 |
| acids | | | | | |
| Phenolic acids | Trace | 0.07 | 0.08 | 0.56 | 50.75 |
| liberated from | | | | | |
| soluble esters | | | | | |
| Phenolic acids | — | Trace | 0.06 | 0.01 | 0.52 |
| liberated from | | | | | |
| soluble | | | | | |
| glycosides | | | | | |

Content of free phenolic acids in the extract from canola meal after hydrolysis (ng/g extract) are listed below:

| Protocatechuic | Caffeic | p-coumaric | Ferulic | Sinapic |
|---|---|---|---|---|
| Trace | 0.11 | 0.81 | 3.64 | 242.08 |

Content of phenolic acids in flaked, cooked canola seeds (mg/g extract are listed below:

| | Protocatechuic | Caffeic | p-coumaric | Ferulic | Sinapic |
|---|---|---|---|---|---|
| Free phenolic | Trace | Trace | Trace | 0.02 | 1.18 |
| acids | | | | | |
| Phenolic acids | Trace | 0.01 | 0.07 | 0.52 | 131.95 |
| liberated from | | | | | |
| soluble esters | | | | | |
| Phenolic acids | — | Trace | Trace | Trace | 0.25 |
| liberated from | | | | | |
| soluble glycosides | | | | | |

### OBJECTS AND SUMMARY OF THE INVENTION

It is an object of the present invention to provide novel therapeutic agents for treating hyperlipidemia.

Another object of the present invention is to provide therapeutic agents for the treatment of hyperlipidemia by lowering lipid levels in blood.

Another object of the present invention is to provide a pharmaceutical composition for treating hyperlipidemia containing, as the active ingredient, at least one canola extract.

Another object of the present invention is to provide a method for treating hyperlipidemia by administering a pharmaceutical composition comprising at least one canola extract.

Another object of the invention is to provide compositions and methods to treat cardiovascular disease, atherosclerosis or hypercholesterolemia, that is, lower cholesterol, apo-B and LDL cholesterol utilizing at least one canola extract to treat a mammal, e.g., a human, at high risk of or suffering from cardiovascular disease.

The above objects and others are achieved by the present invention, which is directed in part to a pharmaceutical composition comprising at least one canola extract effective in treating hyperlipidemia and a pharmaceutically acceptable excipient and methods thereof.

### BRIEF DESCRIPTION OF THE DRAWINGS

FIG. 1 depicts inhibition of HepG2 cell apo B production versus the concentration of total phenolic and tocopherol/sterol extracts.

### DETAILED DESCRIPTION OF THE INVENTION

The present invention is directed to a pharmaceutical composition comprising at least one canola extract effective in reducing elevated levels of lipoproteins and a pharmaceutically acceptable excipient.

In preferred embodiments, the canola extract is selected from the group consisting of a total phenolic, a phenolic acid, a carotenoid, a tocopherol/sterol, a glucosinolate and combinations thereof. In certain embodiments, the combination is a glucosinolate and a phenolic.

The canola extract is incorporated into the formulation in an amount to provide a concentration effective to reduce lipoproteins. The concentration can be, e.g. from about 0.01 ∥g/ml to about 10000 μg/ml. This range is not meant to be limiting as one skilled in the art would be able to determine the effective concentration range to provide the desired effect. The invention is intended to cover any concentration of at least one canola extract which exhibits a reduction in at least one lipoprotein.

In certain embodiments the canola extract may also be administered with another compound capable of lowering blood levels of triglycerides, cholesterol, or glycerol, including but not limited to fibrates (e.g., bezafibrate, gemfibrozil, and clofibrate), HMG-COA reductase inhibitors (e.g., somatostatin, pravastatin, simvastatin, and fluorastatin, atorvastatin, and lovastatin), bile acid binding resins (e.g., cholestyramine and colestipol), nicotinic acid compounds (e.g., nicotinic acid and niceritrol), and fish oils. The additional compound can be administered before, with or after the canola extract.

In certain embodiments, the composition of canola extract comprises a dose of tocopherol/sterol to provide, e.g., a concentration of the tocopherol/sterol from about 0.1 μg/ml to about 500 μg/ml, about 1 μg/ml to about 100 μg/ml, about 5 μg/ml to about 75 μg/ml or a from about 10 μg/ml to about 50 μg/ml.

In certain embodiments, the composition of canola extract comprises a dose of total phenolics to provide, e.g., a concentration from about 0.1 μg/ml to about 1000 μg/ml, from about 1 μg/ml to about 100 μg/ml, from about 5 μg/ml to about 75 μg/ml or from about 10 μg/ml to about 50 μg/ml.

In embodiments where the canola extract comprises a glucosinolate, the glucosinolate can be selected from the group consisting of progoitrin, sinigrin, glucoraphanin, napoleferin, glucoalyssin, gluconapin, 4-hydroxybrassicin, glucobrassicanapin, glucobrassicin, gluconasturtin, 4-methoxy-glucobrassicin, neoglucobrassicin and combinations thereof.

In certain embodiments, the pharmaceutical compositions of the present invention reduce elevated lipoproteins by about 25% or more, by about 50% or more or about 75% or more, depending on the individual needs of the patient.

In certain embodiments of the invention the canola extract is extracted from heat-deactivated canola seeds, canola meal, decolorization by-product, or deodorization by-product and the extraction is by homogenization, centrifugation, precipitation, and vacuum evaporation.

As used herein, the term hyperlipidemia may include both hypercholesterolemia and hypertriglyceremia, and hence, compounds having a hypolipidemic effect may exhibit activity to lower both cholesterol and triglyceride lipid levels.

The invention is further directed to methods of treating a mammal (e.g. a human patient) suffering from hyperlipidemia comprising administering a canola extract effective to reducing at least one lipoprotein. Preferably, the extract is in the form of a pharmaceutical composition as disclosed herein.

The present invention can be administered intravenously, intraperitoneally, subcutaneously, intramuscularly, intrathecally, orally, rectally, topically or by aerosol.

Formulations suitable for oral administration include liquid solutions of the active compound dissolved in diluents such as saline, water or PEG 400; capsules or tablets, each containing a predetermined amount of the active agent as solid, granules or gelatin; suspensions in an approximate medium; and emulsions.

Formulations suitable for parenteral administration include aqueous and non-aqueous isotonic sterile solutions, which contain buffers, antioxidants and preservatives. The formulations may be in unit dose or multi-dose sealed containers.

Dosage amount and interval may be adjusted individually to provide plasma levels of the canola extract which are sufficient to maintain the decreased lipid levels.

Alternatively, one may administer the compound in a depot or sustained release formulation.

A variety of delivery systems for the pharmacological compounds may be employed, including, but not limited to, liposomes and emulsions. The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

### EXAMPLES

A. Extraction of Total Phenolics from Canola Meal

A 2 g sample of canola meal was homogenized in room temp. with 20 mL methanol/water (70:30, v/v). The sample was centrifuged for 10 min, 5,000 rpm and supernatant was collected. The precipitate was extracted 2 more times with fresh portions of methanol/water (70:30, v/v). Combined supernatants were evaporated at 40-45° C. under vacuum and then under nitrogen gas to dryness. For experiments, stock solution (50 mg/mL) was made in DMSO.

B. Extraction of Phenolic Acids from Canola Meal

A 1 g sample of canola meal was homogenized with 20 mL methanol-acetone-water solvent system (7:7:6, v/v/v) for 15 seconds, 10,000 rpm. Extraction was repeated two more times using fresh 1 g samples of canola meal. Combined samples were centrifuged for 15 min, 5,000 rpm. Supernatant was collected and precipitate was extracted 2 more times with fresh portions of methanol/acetone/water. Combined supernatants (˜120 mL from both extractions) were evaporated first at 40-45° C. under vacuum and then under nitrogen gas until the volume reached approximately 20 mL. At this point, 15 mL 4 N NaOH was added and the solution was flushed with nitrogen gas for 4 hours at room temperature. The solution was then acidified to pH 2 using 6 N HCl, transferred to a large saponification tube and extracted 3 times by shaking virgously for 30 seconds with fresh 20 mL portions of diethyl ether-ethyl acetate (1:1, v/v). Combined ether extracts were evaporated to dryness under nitrogen gas. For experiments, stock solution (50 mg/mL) was made in DMSO.

C. Extraction of Carotenoids from Spent Bleach Clay

2 g sample of spent bleach clay was homogenized with 20 mL hexane:acetone:petroleum ether (2:1:1, v/v) at room temp for 1 min and centrifuged for 10 min, 5,000 rpm. Supernatant was collected and precipitate was extracted one more time using a fresh portion of the solvent system. Combined supernatants were protected from light and evaporated under nitrogen gas until a stable volume was reached. For experiments, stock solution (200 mg/mL) was made in DMSO. The highest concentration of carotenoid fraction used was 2 mg extract/mL medium (=0.8% DMSO). To determine whether the extract contained carotenoids, a TLC plate was developed in heptane-benzene (9:1, v/v) along with pure carotenoids (beta-carotene or mixture) as standards. Tested sample produced yellow spots at R_{f} 0.2-0.3, confirming presence of carotenoids.

D. Extraction of Tocopherols/Sterols from Deodorizer Distillate

A 2 g sample of deodorizer distillate was homogenized at room temperature with 20 mL methanol for 1 min and centrifuged for 10 min, 5,000 rpm. Extraction was repeated with fresh portion of hot methanol (to extract sterols). Combined supernatants were evaporated to dryness under nitrogen gas. For experiments, stock solution (200 mg/mL) was made in DMSO.

E. Extraction of Total Phenolics and Glucosinolates from Flaked, Cooked Canola

A 2 g sample of flaked, cooked canola seeds was homogenized at room temp for 1 min. with 6 mL methanol/water (90:10, v/v) and centrifuged for 10 min, 5,000 rpm. Extraction was repeated one more time with a fresh portion of methanol/water. Combined supernatants were evaporated to dryness under nitrogen gas. For experiments, stock solution (70 mg/mL) was made in DMSO.

F. Screening of Extracts

Extracts were tested for cholesterol-lowering potential using human hepatoma HepG2 cells. These cells are known to secrete and catabolize lipoproteins similar to LDL and have been used as a model of human liver. Changes in the medium content of these lipoproteins caused by canola extracts were determined by measuring medium concentration of LDL-associated protein, apo B.

Cells were grown in plates, in minimum essential medium (MEM) containing 10% fetal bovine serum (FBS). Before each experiment, nearly confluent cells were preincubated for 24 hours with MEM containing 1% bovine serum albumin (BSA) instead of FBS.

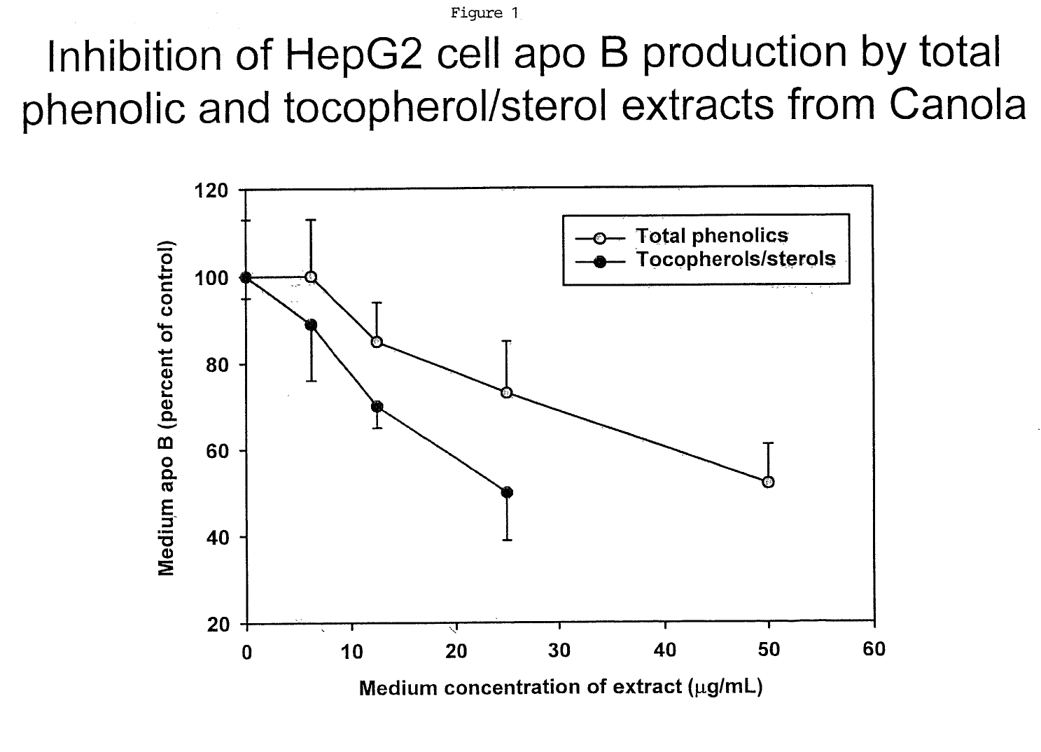

After preincubation, cells were incubated for 24 hours in MEM containing BSA in the absence or presence of various non-toxic concentrations of canola extracts (as determined by MTT viability assay). The apo B content of the medium was then determined by an enzyme-linked immunosorbent assay (Elisa). Cells were washed and dissolved in 0.1 N NaOH for protein determination. The apo B content of the medium was calculated in μg per mg cell protein and expressed as percent of control (medium of cells incubated with DMSO). The results are shown in FIG. 1 and the charts below:

Analysis of effect on medium apo B

| | Concentration | |
|---|---|---|
| Extract | μg/mL medium | % apo B in medium |
| #1 Total phenolics | 50 | 61 |
| | 25 | 75 |
| | 12.5 | 84 |
| | 6.25 | 100 |
| #2 Phenolic acids | 100 | 100 |
| #3 Carotenoids | 3.13 | 100 |
| #4 Tocopherols/sterols | 25 | 50 |
| | 12.5 | 70 |
| | 6.25 | 89 |
| | 3.13 | 91 |
| #5 Phenolics/glucosinolates | 12.5 | 100 |

Serum Lipid Profile In Hamsters Fed 2% Canola Phenolics vs. Control Diet

Serum Lipid Profile In Hamsters Fed 2% Canola Phenolics vs. Control Diet

| | | VLDL + | | VLDL + | |
|---|---|---|---|---|---|
| | Total | LDL | HDL | LDL/HDL | |
| | cholesterol | cholesterol | cholesterol | cholesterol | ^{Triacylglycerlos} |
| | Mol/L | 1 mMol/L | mMol/L | ratio | mMol/L |
| Control | 4.05 ± 0.45 | 2.00 ± 0.16 | 2.05 ± 0.25 | 0.99 ± 0.09 | 5.01 ± 2.23 |
| (7) | | | | | |
| 2% phenolics | 4.41 ± 0.58 | 2.53 ± 0.49 | 1.87 ± 0.15 | 1.35 ± 0.24 | 3.58 ± 0.83 |
| (8) | | | | | |
| Percent | | | −9% | | −28% |
| difference | | | | | |

What is claimed is:

1. A pharmaceutical composition comprising at least one canola extract effective in reducing elevated levels of at least one lipoprotein and a pharmaceutically acceptable excipient.

2. The composition of claim 1, wherein the canola extract is selected from the group consisting of a total phenolic, a phenolic acid, a carotenoid, a tocopherol/sterol, a glucosinolate and combinations thereof.

3. The composition of claim 1, wherein the canola extract comprises a tocopherol/sterol.

4. The composition of claim 1, wherein the canola extract comprises a is total phenolic.

5. The composition of claim 1, wherein the canola extract comprises a glucosinolate.

6. The composition of claim 1, wherein the canola extract comprises a glucosinolate selected from the group consisting of progoitrin, sinigrin, glucoraphanin, napoleferin, glucoalyssin, gluconapin, 4-hydroxybrassicin, glucobrassicanapin, glucobrassicin, gluconasturtin, 4-methoxy-glucobrassicin, neoglucobrassicin and combinations thereof.

7. The composition of claim 1, which inhibits at least one lipoprotein by about 25% or more.

8. The composition of claim 1, which inhibits at least one lipoprotein by about 50% or more.

9. The composition of claim 1, which inhibits at least one lipoprotein by about 75% or more.

10. The composition of claim 1, wherein said lipoprotein contains an apoprotein.

11. The composition of claim 10, wherein said apoprotein is apo B.

12. The composition of claim 3, wherein said composition contains a dose of said tocopherols/sterols to provide a concentration of from about 1 μg/ml to about 100 μg/ml.

13. The composition of claim 12, wherein said composition contains a dose of said tocopherols/sterols to provide a concentration of from about 5 μg/ml to about 75 μg/ml.

14. The composition of claim 13, wherein said composition contains a dose of said tocopherols/sterols to provide a concentration of from about 10 μg/ml to about 50 μg/ml.

15. The composition of claim 4, wherein said composition contains a dose of said total phenolic to provide a concentration of from about 1 μg/ml to about 100 μg/ml.

16. The composition of claim 15, wherein said composition contains a dose of said total phenolic to provide a concentration of from about 5 μg/ml to about 75 μg/ml.

17. The composition of claim 16, wherein said composition contains a dose of said total phenolic to provide a concentration of from about 10 μg/ml to about 50 μg/ml.

18. The composition of claim 1, wherein said composition is suitable for intravenous, intraperitoneal, subcutaneous, intramuscular, intrathecal, oral, rectal, topical or aerosol administration.

19. The composition of claim 1 further comprising at least one additional therapeutic agent for treating elevated levels of lipoproteins.

20. A method of treating a human at risk of or suffering from hyperlipidemia comprising administering a compound of claim 1.

21. A method of reducing elevated levels of at least one lipoproteins in a human patient comprising administering an effective amount of tocopherols/sterols to provide a concentration of from about 1 μg/ml to about 100 μg/ml.

22. The method of claim 21, wherein said composition contains a dose of said tocopherols/sterols to provide a concentration of from about 5 μg/ml to about 75 μg/ml.

23. The method of claim 21, wherein said composition contains a dose of said tocopherols/sterols to provide a concentration of from about 10 μg/ml to about 50 μg/ml.

24. A method of reducing elevated levels of at lipoproteins in a human patient comprising administering an effective amount of total phenolic to provide a concentration of from about 1 μg/ml to about 100 μg/ml.

25. The method of claim 24, wherein said composition contains an amount of said total phenolic to provide a concentration of from about 5 μg/ml to about 75 μg/ml.

26. The method of claim 24, wherein said composition contains an amount of said total phenolic to provide a concentration of from about 10 μg/ml to about 50 μg/ml.
